(19) United States
(12) Patent Application Publication
(10) Pub. No.: US 2005/0112674 A1
(43) Pub. Date: May 26, 2005

(54) Parallel reactor

(75) Inventors: Yoram Palti, Haifa (IL); Daphna Palti-Wasserman, Haifa (IL)

Correspondence Address: DARBY & DARBY P.C., P. O. BOX 5257, NEW YORK, NY, 10150-5257, US

(73) Assignee: Summitech, Ltd., Road Town (GB)

(21) Appl. No.: 10/988,429

(22) Filed: Nov. 12, 2004

Related U.S. Application Data
(60) Provisional application No. 60/523,888, filed on Nov. 19, 2003.

(51) Int. Cl. C12Q 1/68; G01N 33/53; C12M 1/34

(52) U.S. Cl. 435/6; 435/7.1; 435/287.2

(57) ABSTRACT

The present disclosure relates to chemical libraries for identifying receptor-ligand. interactions. It is directed to an apparatus for the diagnosis, recognition, separation, and synthesis of a variety of chemical and biochemical processes, and further provides methods for preparing the apparatus, and processes for the recognition, separation, and synthesis of a variety of chemical, biochemical, and biological processes and substances using the apparatus.

Under 35 U.S.C. § 119(e), this application claims the benefit of prior U.S. Provisional Application No. 60/523,888, filed Nov. 19, 2003, which is incorporated herein by reference in its entirety.

### FIELD OF THE INVENTION

The present invention relates to chemical libraries for identifying receptor-ligand interactions. It is directed to an apparatus for the diagnosis, recognition, separation, and synthesis of a variety of chemical and biochemical processes, and further provides methods for preparing the apparatus and processes for the recognition, separation, and synthesis of a variety of chemical, biochemical, and biological processes and substances using the apparatus.

### BACKGROUND OF THE INVENTION

Numerous chemical, biochemical, and biological processes involving recognition, identification, analysis, selection, isolation, synthesis, and polymerization can take place on an array (microarray) of reaction sites. These sites may be specifically addressed electrically or optically to detect the biological process. The array technology is a powerful tool to identify or process numerous elements, e.g., in high throughput screening (HTS) or processing. Microarray assays enable massive parallel data acquisition, analysis, synthesis and frequently separation, thus making HTS possible. Parallel processing greatly increases the throughput and allows rapid screening, comparison of relative binding affinity, and quantitative assessment of product and reaction rates & yields. For example, genes or gene products represented in the microarray and microarrays of complementary DNA (cDNA) sequences allow hybridization-based monitoring of gene expression. Devices couple an immobilized molecular recognition element, i.e., cDNA nucleic acid, to the surface of a transducer which will “transduce” a molecular recognition event into a measurable signal, pinpointing the presence of the target.

Biosensors that include high density probe immobilization are converging with microfluidic technology to assist in the elaboration of self-contained analytical biochip module systems. The key players who have entered the race to produce primarily DNA chips use techniques based essentially on immobilization chemistries, each with advantages and limitations of its own. So far, these techniques include DNA chips for nucleic acid hybridization Drmanac, et al., Science, 19093, 260:1649-1652: Gingeras, et al., Nucleic Acids Res., 1987, 15:5373-90; Meinkoth and Wahl, Anal. Biochem., 1984, 138:267-284; and sequencing as well as identification of target DNA sequences (Merel, et al., Clin. Chem., 1996, 43: 1285-1286; Yershov, et al., Proc. Natl. Acad. Sci. USA, 1996, 93:4913-4918). These approaches can be divided into two directions: synthesis of the probes directly onto the chip; and direct conjugation of cDNAs or cRNAs to the chip surface.

Current microarray assays focus on nucleic acid hybridization, but microarray and combinatorial chemistry technologies include parallel analysis of proteins, lipids, carbohydrates and small molecules (PCT Publication Nos. WO 99/41006, WO 96/36436, WO 96/24061, WO 98/46548, WO 99/19344, WO 98/11036, WO 97/14814, WO 95/32425). Thus, the high specificity and affinity of biomaterials to their recognition partners, e.g., enzyme-substrate, antigen-antibody, oligonucleotide-DNA, hormone-receptor, etc. permit design of highly specific and sensitive sensor systems.

In the case of microarrays involved in synthesis, the microarrays are prepared in a stepwise fashion by the in situ synthesis of nucleic acids, peptides, and other biopolymers from biochemical monomer building blocks. With each round of synthesis, an additional monomer is added to growing chains until the desired length is achieved. Alternatively, pre-assembled biochemical substances, such as cDNA, which were amplified by PCR and purified, or peptides are conjugated (covalently or non-covalently) onto known chip locations using a variety of delivery technologies.

In order to enable parallel processing, each site on the array (which may include thousands of sites) must be loaded with a different specific molecule, or a series of components. Furthermore, each position should be easily addressable so that the reactions that take place on each site may be monitored. Obviously a closely packed microarray makes the above more difficult to achieve. However, technology increasingly permits higher density arrays, e.g., 10,000 entities in one cm² area.

One type of array, an electrical array, consists of metal (gold, platinum, etc.) electrodes or sites, on which the reaction takes place. Each site is connected to a conductive lead that terminates, for example, in a pad that can be addressed by an electrical connector. The electrical connection can be used for the application of electrical voltage or current to the site, thus enabling the measurement of biological/biochemical invoked changes at the site (generation of voltage differences, currents or changes in impedance, for example). Furthermore, the electric signal can influence the reaction taking place at the site. The above arrays are generally fabricated using microchip technologies, which allow manufacturing of miniaturized systems in large quantities at low cost. These technologies also allow the incorporation of electronic elements such as amplifiers, FET's or photo-diodes to aid sensing. In view of the predicted central role of microarrays in biomedical research, particularly in pharmacogenomics and pharmacoproteonomics, biochip revenues may well eventually eclipse those of computer chips. These predictions are independent of the expectation that powerful computers of the future may harness biological processes to perform logical operations. See also U.S. Pat. Nos. 6,350,368 and 5,942,388 and Drummond et al., Nature Biotechnology, 21, 1192 (2003).

Optical arrays, which are less common, involve sites located at the tip of optical fibers or light guides that replace the electrically conductive elements described above (see PCT Publication No. WO 99/18434).

In the case of microarrays designed for recognition, the sites are in contact with electrodes, and the information generated by the reaction at the site may be either electrical or optical in nature. When this information is of an electrical nature (change in potential, resistance, capacity, electrical current generation, etc.), it is transferred to a control and analysis system by means of the conducting leads and a connector. When the information is optical, for example, the fluorescence peptide/DNA biochips developed by Affymetrix, the information may be transferred to the control and analysis system by optical fibers, or may be remotely monitored, for example, by a CCD camera in combination with a confocal microscope. Multi-color fluorescence allows comparisons of a few samples, e.g., normal and diseased, or diseased and treated, to be made on a single chip.

In all types of arrays loading of each of the sites with the specific designated component or series of components represents a difficult, time consuming and expensive step. Targeting of distinct molecular probes to a particular location on an array at a high density involves delicate, time consuming and precise reagent handling capabilities. Furthermore, the proximity of the different individual array elements, amplify the problem of covalent spatial allotment and their conceivable cross-contamination during their elaboration. The avoidance of the above significantly increases the cost of the process.

A variety of high accuracy placement technologies, which include the use of sophisticated robotic techniques, were developed for this purpose. These include mechanical systems, for example, computerized x-y stages, ink-jet component spray at specific locations, various masks that prevent the reactants to reach any site except the desired one, etc. Electrical systems are also available, for example, electrical currents are used to direct the required constituents, for example, oligodeoxynucleotide probes, onto the activated electrodes with a concomitant increase in the hybridization speed (Heller, 1995, 1996, 1997). However, electronic activation of a single microelectrode cannot fully prevent the remaining electrodes from being reached by the specific DNA probes, with possible cross-contamination.

Random combinatorial libraries of molecules avoid the difficulties of site-specific array development by obviating the requirement for precise reagent disbursement and handling. Various combinatorial library technologies have been developed, such as, but not limited to split synthesis libraries (see PCT Publication Nos. W092/00252, and WO 94/28028), as well as various natural product and synthetic libraries (discussed below). However, the ease of synthesis or creation of these libraries includes a cost: their randomness may result in uncertainty about the structure of a molecule in the library. This can also be an issue in directed placement arrays as well. This problem is addressed in more detail below. These molecular arrays and libraries on solid phase supports share a more basic technical feature as well.

Both molecular arrays and combinatorial libraries on solid supports require immobilization technologies that secure the molecules, permit biological processes to occur, and preferably allow repeated use of the array or support. A common technology for immobilization of the desired specific component on the selected specific site or a support involves masking of all sites, except for the desired one, and then applying the desired component to the whole surface. The component will react and be immobilized only at the exposed site. The masking may be done, for example, by shining an IR beam only on the site of interest. The heat thus generated locally melts the mask and the site is exposed. The site should be saturated so that further binding does not occur. However, cross-contamination, i.e., binding of undesired components onto a previously exposed site can not be absolutely prevented.

Binding, as well as other reactions, may be enhanced by applying an electrical potential or illuminating the sites.

While array manufacturing can be difficult and expensive, identification of the structure of a molecule, whether the sequence of an oligonucleotide or peptide, substituents on a pharmacore, or new variations in substitutents or structure, is often difficult. Microsequencing techniques and nucleic acid amplification permit direct sequencing of nucleic acids and peptides. However, direct sequencing is expensive, difficult, and time consuming. Early work with “tag ged” libraries used chemical tags as surrogate identifiers of the molecule. These include peptides or oligonucleotides (See PCT Publication Nos. WO 93/06121, WO 94/28028, and WO 97/00887), radioisotope-labeled compounds (see PCT Publication No. WO 97/14814), and fluorine tags (see, PCT Publication No. WO 99/19344). These strategies suffer from the possibility that the tags themselves contribute to the biological process measured in the assay.

Alternative technologies employ spectroscopic tags that uniquely identify molecules in a library. Examples of such tags include infrared/Raman spectroscopic tags (PCT Publication No. WO 98/11036), and fluorescent labels (PCT Publication No. WO 95/32425). Nuclear magnetic resonance spectrometry permits elucidation of fluorine and ¹³C/¹⁵N tags (PCT Publication Nos. WO 99/19344 and WO 97/14814).

Convergence of computer data storage and microarray/molecular library technologies has recently yielded alternatives to chemical or spectroscopic identification techniques. These approaches include using silicon chips with embedded machine readable codes (PCT Publication Nos. WO 99/41006 and WO 96/24061), and matrix materials with programmable data storage or recording capacity (PCT Publication No. WO 96/36436).

However, these strategies, like addressable microarrays, depend on implementation of complex and expensive technologies. As a consequence, these techniques cannot achieve mass market potential for medical laboratory diagnostic use or routine forensic testing. There remains a need in the art for affordable technology for identification of molecular structures on arrays and combinatorial libraries. The present invention advantageously addresses these and other needs in the art.

### SUMMARY OF THE INVENTION

The present invention provides a solid-support apparatus useful for the recognition, separation, and synthesis of a variety of chemical, biochemical, and biological processes and substances, including those occurring within living subjects. The apparatus comprises a series of individual perforated elements assembled to form a perforated element stack or a plurality of perforated element stacks, wherein the perforations of each perforated element overlap to form a central tunnel or tunnels through which one or more substrates may flow.

In one embodiment, a molecule, such as a nucleic acid, a carbohydrate, an oligonucleotide, a peptide, a peptideomimetic, a pharmacore, a biosensor, or a antibody is immobilized on the inner surface of at least one of the perforations within a stack.

In a second embodiment, a plurality of perforated element stacks are combined to form a solid support library.

A third embodiment is directed to a process for preparing the solid support and solid support library.

In a fourth embodiment, a method of identifying a molecule of interest within the solid support by reading of information from the different sites on the perforated elements is set forth.

The advantages of the present invention, when compared to the existing devices such as micro arrays and biochips, include low operating costs, maximum flexibility, high throughput, and the ability to use very small amounts of substrates for all steps of the process of construction and use. Furthermore, the unique reading procedure set forth herein offers a much less complicated and a less expensive alternative to the currently available micro-array reading devices.

### BRIEF DESCRIPTION OF THE DRAWINGS

FIGS. 1A-D show individual perforated elements of different shapes. FIG. 1E is a cross section of the element of FIG. 1A, showing the interior perforation of the element coated with a selected molecule(s) to be used as a support and reactive element on the site.

FIG. 2A shows individual perforated elements stacked to form a single tunnel. FIG. 2B shows individual perforated elements stacked to form a single tunnel, using separating elements.

FIG. 3 shows several stacked columns joined to form a 3-D matrix of individual perforated element stacks.

FIG. 4A shows activation of the sites by moving a substance including one or more substrates to be detected or activated by pushing the substance through a tube created by stacked elements using a piston. FIG. 4B shows activation of the sites by moving the sample through the stack using fluid flow.

FIG. 5 shows an approach for reading from the different sites, whereby a reading probe is threaded through the tube created by the stacked elements.

FIG. 6A shows optically illuminating and reading from a single stack, using a split bundle of optical fibers and a conical prism. FIG. 6B shows optically illuminating and reading from a single stack using a split bundle of optical fibers and a mirror positioned at an angle to the bundle.

FIG. 7 shows an excitation and emission light pathway for the embodiment described in FIG. 6A.

FIG. 8 shows a partitioned stack of elements.

FIG. 9 shows a stacked micro-lab, based on external reading from the stack.

FIG. 10 shows a stacked micro-lab, based on external reading from the stack, whereby light slots are used to guide light signals in and out of the stack.

FIG. 11 depicts the use of the stacked elements for in line real time monitoring of fluid content.

FIG. 12 depicts an application of the in line real time monitoring stack whereby the stack is implanted into a living body, and used to monitor fluid content.

FIG. 13 depicts an application of the stack whereby a capillary stack of elements is used to acquire and test a blood or urine sample.

FIG. 14 shows a multi-inlet and/or multi-outlet stack of elements.

FIG. 15 depicts data acquisition by measurement of electric potential differences.

### DETAILED DESCRIPTION OF THE INVENTION

The present invention is designed to enable high throughput diagnosis, recognition, separation, and synthesis of a variety of chemical, biochemical, and biological processes and substances by means of a solid-support apparatus constructed from one or more perforated element (PE) that form an assembly of support sites.

The apparatus consists of perforated elements that serve as the building blocks for the device. These perforated elements are used to create one or more stacks of perforated elements, through which a sample is moved. By column stacking the individual elements, one or more tube-like tunnels or cavities are formed within each stack. These individual elements may be stacked in many different ways to form a variety of one-, two-, or three-dimensional structures. The terms cavity and tunnel may be used interchangeably throughout this disclosure, and are defined herein as the central opening(s) that run internally through the length of each stack, formed by overlap of the one or more perforations within each perforated element.

Each element is identified by its location in the sequence of elements within the stack. This location determination can be aided by periodic introduction of reference elements throughout the stack. In one specific embodiment, the individual PE's support the different processes and substances on the interior face of the perforation in the element.

Placement of the substances involved in the reactions as well as the activation of the sites is achieved by moving a very small amount of fluid along the inner tube-like cavity that is formed when the individual PEs are combined. The sample may be introduced into the stack of PEs and remain there for the duration of the reaction, or it may be made to flow along the stack. The resulting reaction, or sequence of reactions, produces a product that can be detected by measuring, for example, the resulting fluorescent light emission or electrical potential generated, or by measuring changes in impedance. Coupled reactions may also be achieved using the stacks. For example, the reaction products from one PE can take part in the reaction taking place on the next PE in the stack. The next PE in the stack is determined by the direction of flow of the sample through the stack. The flow direction can be alternated.

The present invention also provides a method for preparing the PE stacks, and for reading them.

### The Perforated Elements

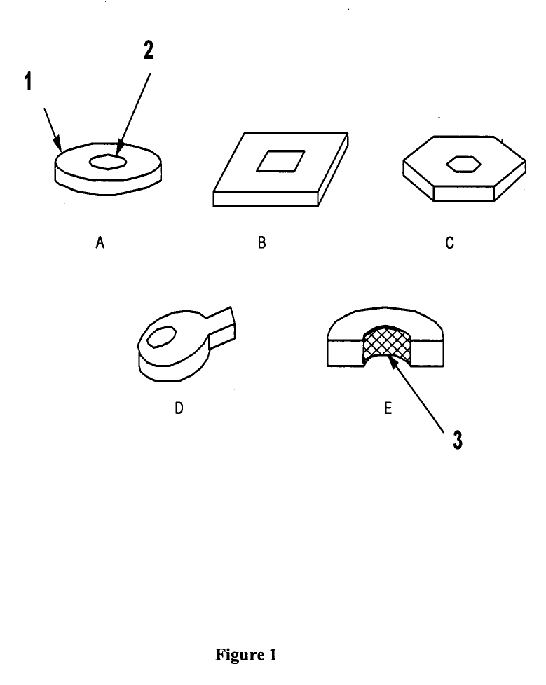

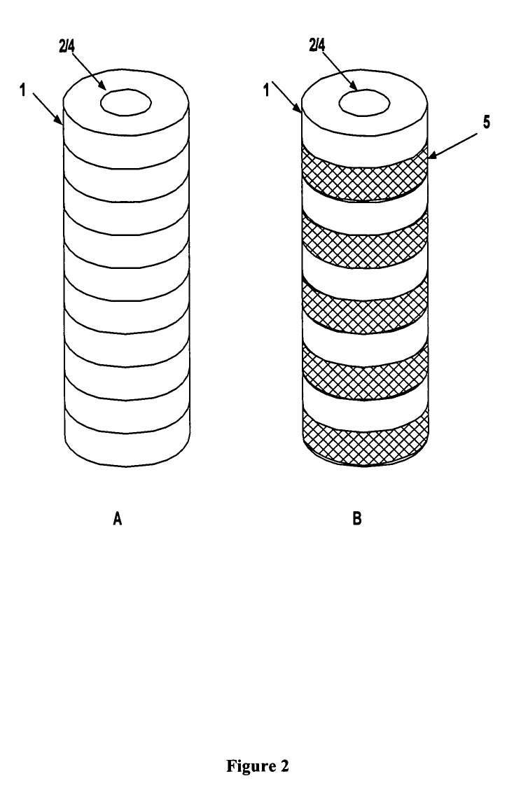

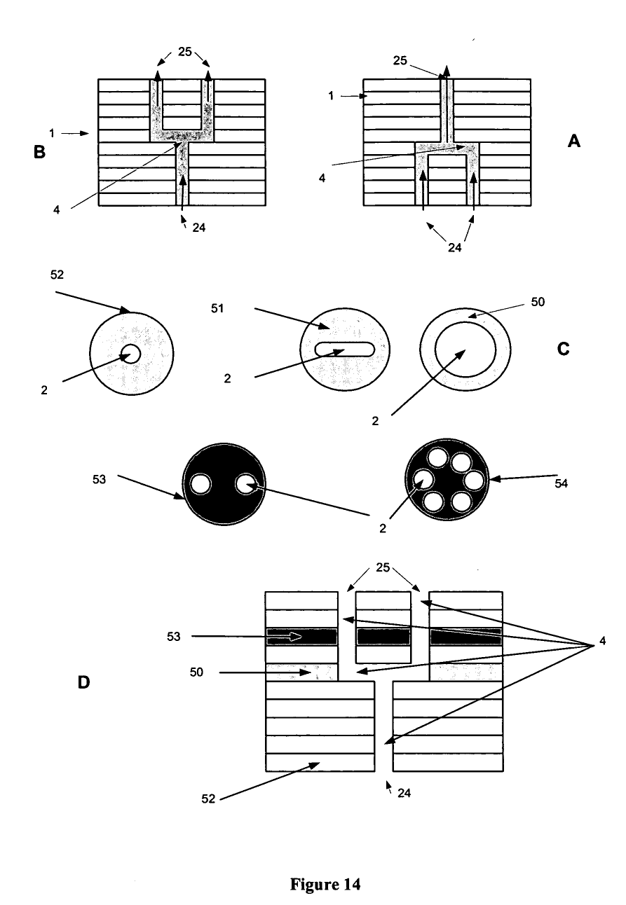

The individual perforated elements (element 1 in FIG. 1) may be of any geometric shape, provided they contain a perforation (element 2 in FIG. 1), and may be asymmetrical or symmetrical with reference to their central axis (see FIGS. 1A-D). Suitable geometric shapes include, but are not limited to, a circle, an equilateral triangle, an isosceles triangle, a scalene triangle, a square, a rectangle, a rhombus, a parallelogram, a trapezoid, a quadrilateral, a hexagon, and an octagon. Each perforated element may also contain more than one perforation (see FIG. 14). The perforation(s) may be the same shape as the PE or different. For example, a circular PE may contain a square perforation, etc. The PE's may be made of a variety of materials. The choice of construction material will be readily apparent to one skilled in the art, and will be determined according to the specific application to be practiced.

Suitable construction materials include, but are not limited to, glass, metals, such as titanium, silver, gold or platinum; organic polymers, such as polystyrene, polyethylene, polypropylene, polymethyl methacrylate (PMMA, acrylic plastic), copolymers of styrene and acrylic acid (PS/PAA), copolymers of styrene and methyl methacrylate (PS/PMMA); polycarbonates, such as Bisphenol A; semiconductors, such as silicon, silicon carbide, and indium nitride; polyurethanes; polyacrylamides; silica; cellulose derivatives, such as cellulose acetate; ceramics, such as machined alumina, zirconia, silicon carbide, and silicon nitride; and combinations thereof.

In addition, the material for the perforated element may be chosen for its functional characteristics. For example, the elements may be magnetic for ease of handling; they may be electrically conductive, insulated, resistive to enable heating by current flow, or they may be transparent or semi-transparent (they could be narrow band selective (optic filter)) so as to enable the specific optical reading procedures described herein to be carried out.

The size of an individual PE can vary considerably according to the desired application. The maximum distance across the surface defining the shape of the individual PE's may vary typically between from about 100 microns to about 10,000 microns, preferably from about 500 microns to about 2,000 microns. The thickness of the individual PE's may vary from about 10 microns to about 1,000 microns, preferably from about 25 microns to about 200 microns. Each perforation in the PE may vary from about 10 microns to about 5000 microns, preferably from about 50 microns to about 200 microns. For most applications, each individual PE is between about 0.1 and about 10 mm across the shaped surface, about 10 and 500 microns in thickness, and has perforation(s) in the range of about 0.01 to about 4 mm.

Consider a stack of circular shaped perforated elements. The more perforated elements are present in the stack, the thickness of each PE may be made smaller in order to obtain a total stack length of convenient size. Thus, a stack holding on the order of 100 PE's may be constructed using perforated elements 0.1 mm in thickness, thereby creating a stack of perforated elements about 1 cm in length. Stacks made up of a larger number of PE's may use PE's with thicknesses on the order of 10 to 50 microns. Very large stacks may, of course, use even thinner individual PE's. Stacks may also be made up of individual perforated elements that each have different thicknesses. The diameter of the perforation may be 0.1 to 0.5 mm when the available sample volume is large and the reactants are readily available. For other situations, such as when reactants are less available or are more costly, PE's with perforations on the order of 10-50 microns in diameter may be used. The total surface area available on such sites is similar to or smaller than the ones currently in use in micro-chips. The length across the shaped surface of the PE can be as small as 100 microns when device volumes are important (i.e., limited) or may be on the order of a couple of mm in diameter when volume is not important. The larger the diameter of the individual perforated element, the easier the handling and the greater the robustness of the stack. The large diameters PE's, i.e. low resistance to flow, are also useful, when large through flows are necessary. Maintaining a fixed diameter lumen throughout the system will prevent turbulent flow effects.

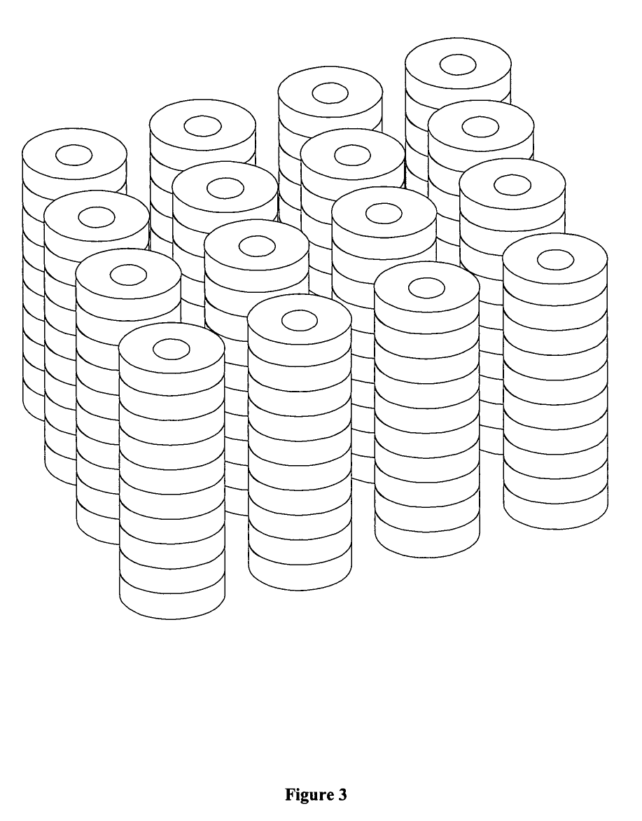

The PE's (element 1 in FIG. 1) are stacked one on top of the other such that their corresponding perforations (element 2 in FIG. 1) form a continuous tube (element 4 in FIG. 2). Each individual perforated element is designed to support a reaction site, process, or reagent, and each site or a few sites can differ from every other, or from neighboring sites. The reaction site may be on the inside or outside surface of each perforated element. In a preferred embodiment the element's reaction support site is located on the inside face of the perforation, i.e. on the interior of each individual element (element 3 in FIG. 1E).

### Coating the Perforated Elements Using a Preparatory Stack

To prepare the PE's for use, their sites must be coated with the appropriate molecule, reagent, or substrate. An efficient method to coat the PE elements involves stacking them, one on top of the other, to form a “Preparatory Stack”. Each Preparatory Stack may be uniformly coated with a single specific substance, or set of substances, without the risk of cross contamination between sites.

The coating of these stacked elements can be done using a variety of methods. For example, liquid containing the coating substance may be flushed along the interior of the Preparatory Stack. The flushing can be performed by flowing fluid along the whole length of the tube, or by passage of a bolus along it, using, for example, force (such as capillary force), a pressure gradient, or a piston. Other suitable methods for coating the stacked elements include, but are not limited to, painting, dipping, spraying, electroplating, subjecting the sites to an electric potential, electric current of selected characteristics, and illumination. The dipping method is more appropriate, when using less expensive coats. Binding of the substances to the PE can be aided by, for example, heating, drying, or flashing of light such as UV. The illumination can be achieved by a device similar to that used for reading optical signals from the stack.

The perforated elements are coated with an appropriate linker, if desired, and then with the appropriate molecules, reagents, or substrates. The danger of “contamination”, i.e., the partial or complete coating of a site by a wrong molecule, is non-existent, as no reactant or type of linker, others than the one intended for use within a particular preparatory stack, are present when the molecule(s) are immobilized on the perforated element reaction sites within a particular preparatory stack.

A “linker” is a moiety, molecule, or group of molecules attached to a solid support. Typically a linker may be bi-functional, wherein the linker has a functional group at each end capable of attaching to a monomer or oligomer, and to a solid support or substrate. The solid support or substrate surface may be modified/coated by one or more linkers including, but not limited to amino, carboxyl, thiol, aldehyde, hydrazide, and combinations thereof for better protein/polynucleotide binding. The polynucleotides are immobilized to the solid substrate or support through covalent bonds, which take advantage of positively charged surface, produced by amino silane or polylysine.

Various reactants or ligands can be attached to the support. These include, but are not limited to, oligonucleotides, peptides, peptideomimetics, pharmacores, antibodies, tumor markers, and biosensors (useful for detecting glucose, cholesterol, ions, and metals).

An “oligonucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix, as well as “protein nucleic acids” (PNA) formed by conjugating bases to an amino acid backbone. This also includes nucleic acids containing modified bases, for example thio-uracil, thio-guanine and fluoro-uracil.

The oligonucleotides may also be modified by many means known in the art. Non-limiting examples of such modifications include, substitution of one or more of the naturally occurring nucleotides with an analog, and internucleotide modifications such as, for example, those with uncharged linkages (such as, but not limited to, methyl phosphonates, phosphotriesters, phosphodiesters, and carbamates) and with charged linkages (such as, but not limited to, phosphorothioates, and phosphorodithioates). Polynucleotides modified by alkylation (covalent binding; such as, but not limited to, metamicine), intercalation, groove binding, electrostatic interactions (non-covalent binding; such as, but not limited to, doxorubicin and acridine), and chelators binding (metal-binding molecules; such as, but not limited to, EDTA and desferrioxamine) may be used for drug discovery.

A “peptide” is a chain of chemical building blocks called amino acids that are linked together by chemical bonds called peptide bonds. Peptidomimetics are compounds structurally mimic peptides but lack the peptide bond.

### The Multi Reaction Stack

Following the coating of the PE elements in the preparatory stack or stacks, the required “Multi Reaction Stack” (MRS) can be constructed. Multi Reaction Stacks are prepared from PE's with different coatings obtained from selected “Preparatory Stacks” having different coatings. Each PE in the MRS acts as a separate active site at which a specific reaction takes place with the fluid sample that is undergoing testing.

The different individually coated PE's can be stacked in many different ways in the MRS, thereby enabling a variety of structures to be formed. The structures differ in their content (the presence and order of different active sites), size, and shape. The present invention allows a variety of structures to be prepared, and thereby provide solutions to a large range of current and future applications and requirements. Repetitions can be used for statistical verification of the response.

In one embodiment, the perforated elements (element 1 in FIG. 1) are stacked one on top of the other, creating a single column of Individual Perforated elements (element 4 in FIG. 2A). The different sites on the PE's are identified by their location along the stack.

In another embodiment, for applications which require a very large number of sites, a matrix of these stacks can be prepared in a similar manner. A 3-D matrix may be created by positioning numerous single stacked columns adjacent to one other (see FIG. 3). Such a matrix may provide an apparatus that can support hundreds of thousand of active sites in a volume under 1 cubic centimeter.

In a further embodiment, the PE's within each stack are separated from each other during the stacking process with perforated separators (element 5 in FIG. 2B). The perforated separators act as spacers between the PE's. The separation may occur by several mechanisms, including, but not limited to, physical, electrical, and optical isolation or insulation. The perforated separators may be identified, for example, by their color, and thereby serve as an aid in determining the active PE position in the stack, and help to avoid errors in determining the location of each individual PE in the MRS.

The construction of the MRS may include means for leakage prevention, thereby ensuring that liquids flushed through the inner tube of the stack do not leak to the outside of the stack. The leak-proofing may be constructed in many ways. For example, each PE surface may be coated with sealing glue that binds to the next PE in the stack. The adhesive coating may be pressure sensitive (self adhesion), heat activated, or UV light activated.

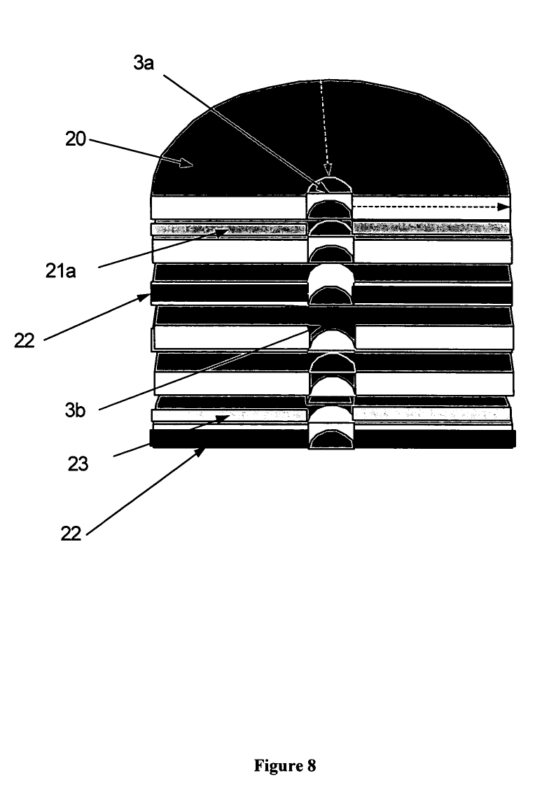

In another embodiment, for applications where specific reagents are needed in order to activate the different sites, a partitioned stack may used (see FIG. 8). In this embodiment, layers of different reagents (elements 21a and 23 in FIG. 8) are embedded near the active sites (elements 3a and 3b in FIG. 8). The different active sites are separated by special partitions (element 22 in FIG. 8) that close when pressure is applied to the area. The disk suface is depicted by element 20 in FIG. 8. Before the sample is flushed into the stack, pressure is applied to specific parts of the stack. Triggered by the pressure, the different reagents are released (one at a time) and the partitions seal a specific volume of the reagents in the stack, thereby allowing the different sites to be activated without the risk of contamination.

### Activation of the Multi Reaction Stack—“Push Through-Processing”

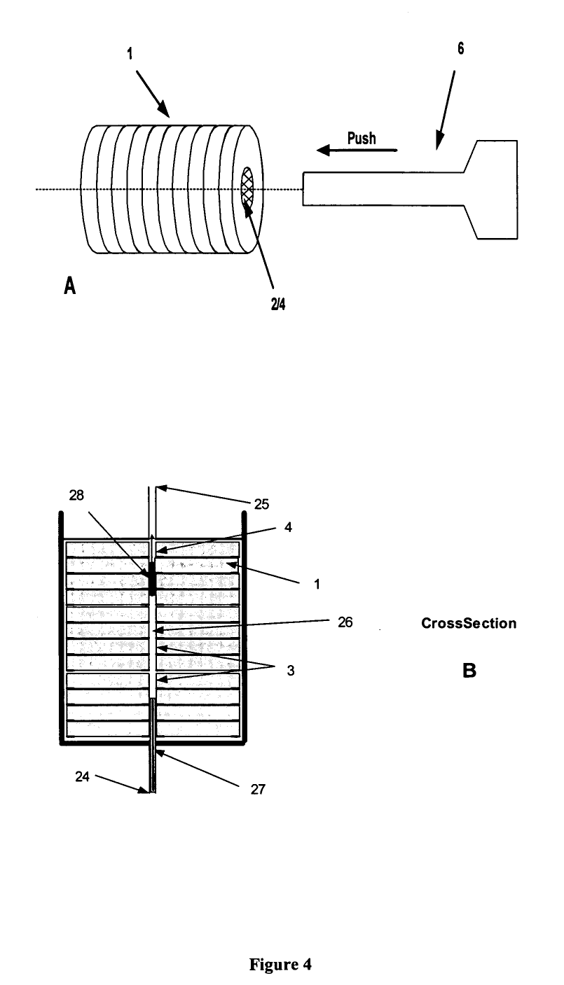

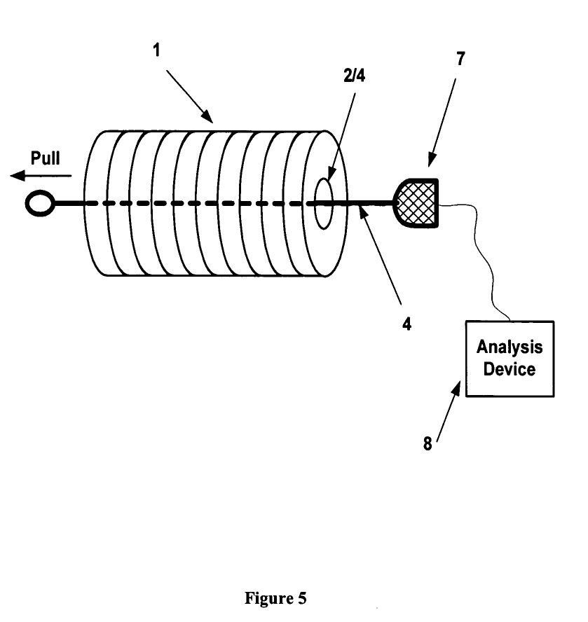

The present invention takes advantage of the morphology of the internal tube created by stacking the different individual elements in the multi reaction stack (see FIG. 2). The activation of the multi reaction stack may be achieved by passing a very small amount of substance, or sample to be tested, etc., through the internal tube (element 4 in FIGS. 4A and B). This may be accomplished in variety of manners. Suitable methods include, but are not limited to, pushing the substance using a piston (element 6 in FIG. 4A), and flushing a small amount of fluid (element 27 in FIG. 4B) through the internal tube by creating a pressure gradient. Capillary forces may also be used for this purpose. The sample may fill the entire stack or a fraction of the stack. If only a bolus of sample is used (element 28 in FIG. 4B), an air bubble (element 26 in FIG. 4B), trapped between the sample and some other liquid, is used to move the sample along the stack (see FIG. 4B). If several substances are to used sequentially on the different sites, they may be moved through the tube one after the other.

The small diameter of the tube, combined with the method of moving the desired substance or substances along the internal tube, enables the use of a very small amount of substrates (on the order of nano-liters to micro-liters) for the fabrication of the PE's, the activation and other processing steps. The length of the sample moving through the stack may be a fraction of the total length of the stack. In such a manner, the present invention provides a simpler, more economical and efficient procedure for activating the different sites, compared to currently known methods.

In a further embodiment, as depicted in FIGS. 14A, 14B, and 14D, a stack may have a plurality of inlet tubes (element 24 in FIG. 14A) or a plurality of outlet tubes (element 25 in FIGS. 14B and 14D) that enable alternate flushing of different fluids. The plurality of inlet tubes converge into one (or more) connecting tubes (element 4 in FIGS. 14A and 14B) by the interposition of a PE with a special perforation in the form of a slit (element 51 in FIG. 14C), that connects the input tubes with the corresponding connecting tube, or by use of a PE with a wide diameter perforation (element 50 in FIG. 14C) that similarly connects inputs tubes to the connecting tube (see FIG. 14C). Such an arrangement can serve, for example, to create duplicate probes or “multiplicate” sensor probes to achieve statistically significant results.

In FIG. 14D, a cross section view of the embodiment of FIG. 14B is detailed. In this embodiment, PE's with a single perforation (element 52 in FIG. 14D) are stacked to form a single inlet tube (element 24 in FIG. 14D). A PE with a large diameter perforation (element 50 in FIG. 14D) creates a junction that results in a connection with two outlet tubes (element 25 in FIG. 14D). The two parallel channels are formed by using PE's with a double perforation (element 53 in FIG. 14D). In another embodiment, more than two outlet tubes may be created by using a PE with multiple perforations (element 54 in FIG. 14C) instead of the double perforation PE.

### Data Acquisition & Analysis Via “Pull Through Reading”

The present invention also provides a method for reading the reaction results obtained using the stacked PE's. The information regarding the reactions at the stacked sites can be in any form. Suitable examples include, but are not limited to, optical (such as fluorescence, opacity, birefringence, and light scattering), and electrical (such as potential differences, electrical currents, and impedance). The reading method is selected according to the form of the data. The information, from the column of elements on which the reactions take place, can be read in one of two basic methods.

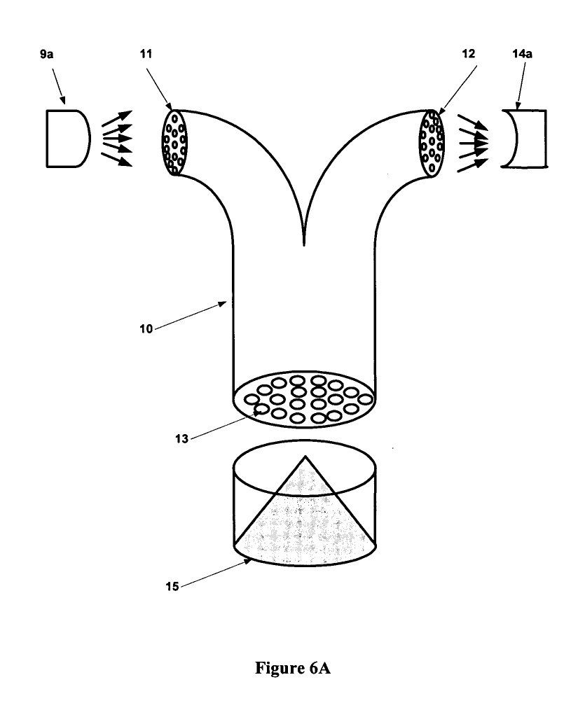

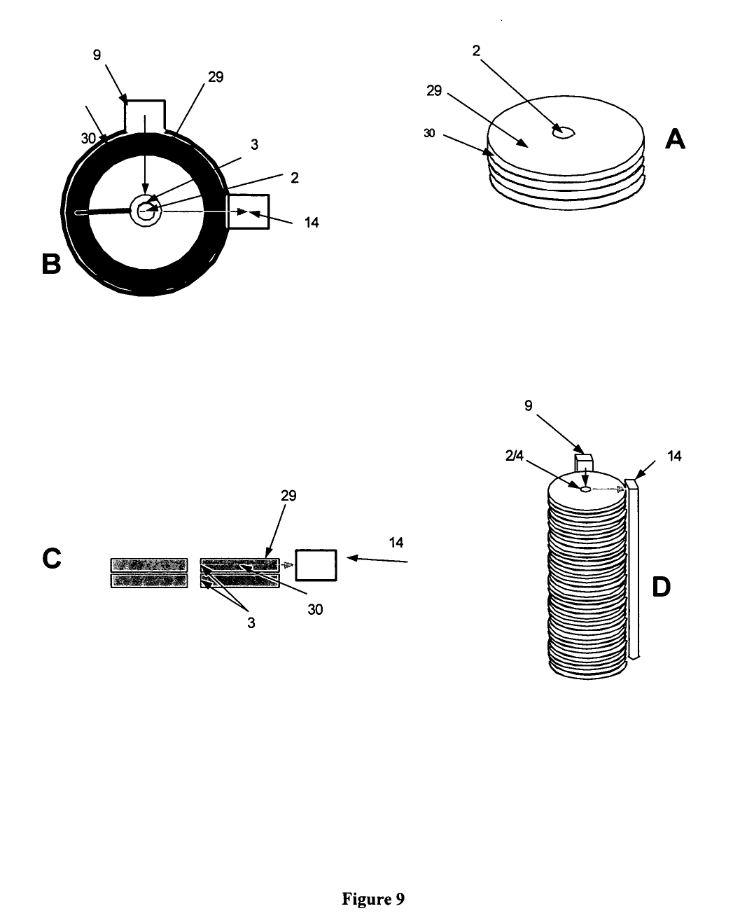

The first embodiment involves an internal reading method (see FIG. 5). In this embodiment, a suitable shaped probe, typically, although not limited to, a thin and/or elongated probe, is threaded along the internal tube created by the stacked elements. The second embodiment involves an external reading method (see FIG. 9). In this embodiment, the stack is scanned from the outside using a probe located externally to the stack. When reading optical information from the stack, both embodiments, i.e., internal and external reading methods, may be used.

In the internal reading method, a probe (element 7 in FIG. 5) is pulled, pushed, or moved with a rotational motion through the internal tube (element 4 in FIG. 5). The movement of the probe may occur manually or by use of a mechanical apparatus. The signals from the different reaction sites along the multi reaction stack are sensed and transmitted sequentially. The signals thus are transmitted to an analysis device (element 8 in FIG. 5) one after the other at a rate determined by the pulling (or pushing) velocity. The reaction sites are identified by their signal's relative position in the sequence of transmitted signals that are received at the analysis device. Identification marks located on the PE's or on the spacer rings along the stack may be used to improve identification of the different PE reaction sites. Suitable identification markings include, but are not limited to, color-coding, magnetic coding, short-circuiting conductors, bar codes, or other optical markings.

The velocity of the probe along the internal tube created by the stacked elements does not affect the quality and robustness of the signal reading. Thus, in contrast to arrays where the differentiation between the sites is extremely dependent on very high and accurate spatial (x,y) resolution, in the present invention, resolution is determined in the time domain. The probe's movement along the inner tube of the stack is constrained by the shape and dimensions of the tube, such that it cannot go “off-track” (see FIG. 5).

The probe itself can be any sensing probe (optical, electrical, etc.) The choice of probe to be used, as readily determined by one skilled in the art, depends on the specified application and the desired requirements.

To prevent the sensing probe from touching the active sites in the internal tube wall, spacer rings may be used that have a central perforation slightly smaller in diameter than the perforation in the perforated elements. In such a manner, the probe is guided away from the active PE's on which the reactions take place. Alternatively, minute extensions in the circumference of the internal tube, may be used to keep the probe at the correct distance from the active sites.

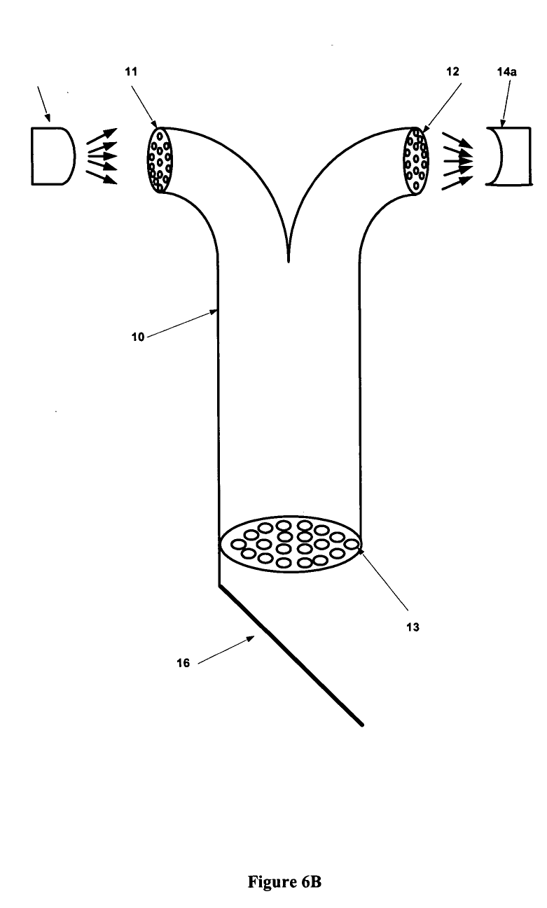

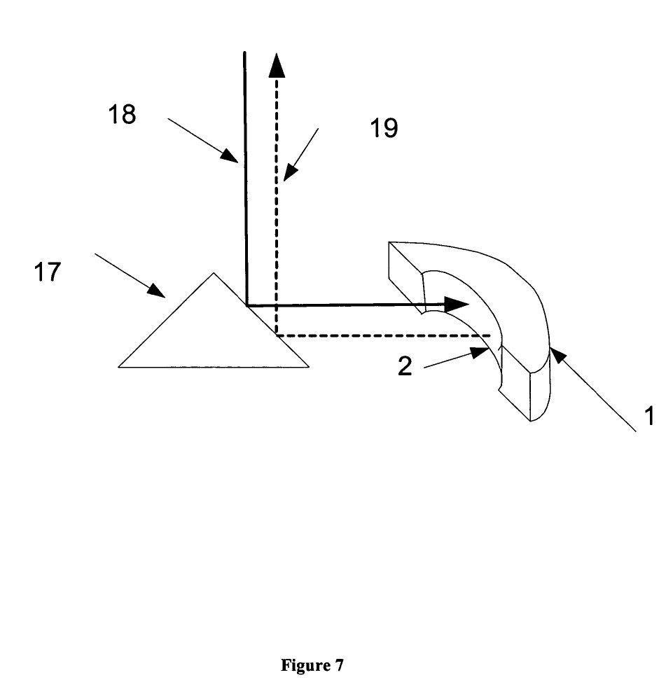

In a specific embodiment, when internal optical sensing is required (see FIGS. 6A and 6B), a detection apparatus which includes an optical fiber bundle (element 10 in FIGS. 6A and 6B) may be used. A single optical fiber is depicted by element 13 in FIGS. 6A and 6B. In a specific embodiment, an incoherent bundle split at one end into two sub-bundles (elements 11 and 12 in FIGS. 6A and 6B) is used. The fibers from one sub-bundle (element 11 in FIGS. 6A and 6B) are used to guide light from an external light source (element 9a in FIG. 6A) to illuminate the sites, while the light, reflected from the interior of the stacked column, travels through the fibers of the other sub-bundle (element 12 in FIGS. 6A and 6B) to an external light-sensing device, such as, but not limited to, a photoelectrical cell, or charge coupled device (CCD) (element 14a in FIGS. 6A and 6B). To enhance light sensing from each PE inner face, which is located in a direction normal to the direction of the light guide, an optical device (element 17 in FIG. 7) may be added at the end of the bundle. For example, a conical prism (element 15 in FIG. 6A) or a diagonally positioned mirror (element 16 in FIG. 6B), may be attached to the end of the optical fiber bundle. Such an optical element helps sense light originating from the different sites by changing the angle of the light, which may be reflected or fluorescent, to an angle that is consistent with light to be guided “up” through the optical fibers to the light sensor (see FIGS. 6 and 7). The fibers from the two sub-bundles can be randomly positioned at the united end of the bundle.

In a further embodiment, when light excitation is required for reactions involving fluorescence, the fiber optical bundle may be split in to two bundles, as described above. The first bundle is used to guide the required excitation light to the interior of the stack, while the second bundle guides the emitted fluorescent light to the sensing probe (see FIGS. 6 and 7). In these applications, light filters and dichotic mirrors may be utilized to enhance and selectively pass the emitted light signals from the sites to the sensing probe.

In FIG. 7, the light path within a single perforated element (element 1 in FIG. 7) is demonstrated. Light (element 18 in FIG. 7) travels from the source to some point in the stack. An optical surface (element 17 in FIG. 7) alters its path by 90 degrees, so it the light reaches the active site on the inner surface of the perforated element (element 2 in FIG. 7) at a right angle. The returned light (element 19 in FIG. 7) similarly travels from the perforated element toward the optical surface, where it changes angle again, and travels to the light sensor/detector.

In yet another embodiment, the optical sensing is achieved by means of a miniature light source (for example, an LED) and a miniature light sensing element that are threaded together along the hollow tube to detect the optical signals from the different reaction sites (see FIG. 5).

When reading the information using the external reading method (see FIG. 9), the probe (element 14 in FIG. 9) is positioned on the outer surface of the stack. The probe may be the same size as the stack or smaller, in which case it slides along the external surface of the stack. For the external reading method, the PE's are constructed in such a manner that they have the ability to transmit the signals to the external surface and thus to the detector.

When using external optical reading, the optical reader is positioned along the outer surface of the stack (see FIGS. 9A and 9D). In this embodiment, the PE's are transparent (element 30 in FIG. 9) and may be coated on their surfaces, that make contact with other PE's, by an opaque layer (element 29 in FIG. 9). Suitable, but non limiting examples of an opaque layer are gold, black colored polystyrene, polycarbonate and ceramic. Alternatively, opaque spacers may be interposed between the perforated elements, so as to prevent light from one element reaching others elements. An external device (element 9 in FIG. 9) illuminates the active sites, through the transparent PE's. The data is read by one or more optical sensors (element 14 in FIG. 9), which scan the stack, or form its image on a sensing linear matrix (such as a CCD) (FIGS. 9B, 9C and 9D). When using transparent PE's, the illumination source may be positioned either outside or inside the stack.

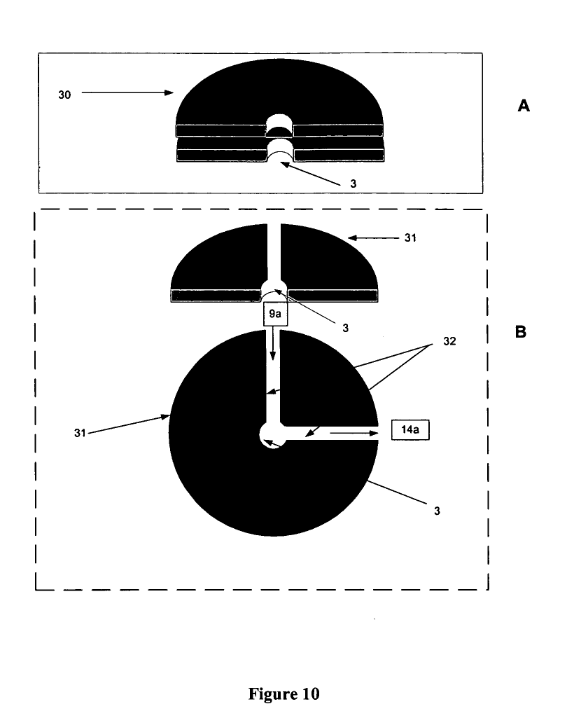

Another embodiment for externally reading the stacks is depicted in FIG. 10B. Element 31 in FIG. 10B depicts a non-transparent disc shaped PE with light slits (element 32 in FIG. 10B)F A perforated element with two light slits is utilized instead of the transparent perforated elements described above. In this embodiment, light from the external light source (element 9a in FIG. 10B) reaches the internal active site (element 3 in FIG. 10B) through a first light slot in the PE (element 32 in FIG. 10B). The detected light reaches the external detector (element 14a in FIG. 10B) by passing through a second light slot (element 32 in FIG. 10B). For practical reasons, the two light slots are filled with wedges, to allow safe flow of the sample and other fluids through the central channel during stack preparation and activation. Only during the reading/analysis stage are these fillings removed to enable the active sites to be optically illuminated and read.

When using an electrical probe to read a multi reactor stack, the stack may be read using either the external reading method (see FIG. 9) or the internal reading method (see FIG. 5). When using an electrical probe, the optical sensor is replaced by a set of electrodes, a sensing coil, or some other electrical sensing device, coupled to a potential, current or impedance measurement circuit located either in the probe or externally. The potential differences, currents, etc. are monitored between two points on the probe, or a point on the probe and another point serving as a common point in the fluid inside the perforation, or outside the stack when some members of the stack are conductive.

When the internal reading method is used, the electrical probe is moved through the inner tube of the stack, in a similar manner to that employed when using an optical probe (see FIG. 5). Electrical reading may also be performed, by using an external probe, or one moving along an additional perforation that runs throughout the PE stack.

In another embodiment, the PE's stacks may comprise PE's made of a conducting material, such as, but not limited to, titanium, silver, and gold, and alternate spacer elements made of an electrically insulating material, such as, but not limited to, polyesters, epoxy laminates, cellulose acetate, phenolic laminates, and silicone. The potential differences, impedances, or currents to be measured, are read by sliding a conducting probe along the stacked PE's, either internally or externally. More specifically, the conducting probe comprises only a section like a ring (the same width as the PE) which is conducting, and only this ring makes contact with the PE's. For practical reasons, in this embodiment, the shaft's core is conducting but it is insulated from the outside. Alternatively, for the external electrical reading method, the conducting PE's may be coated with a non-conducting material, such as, but not limited to rubber, glass, silica, or cellulose.

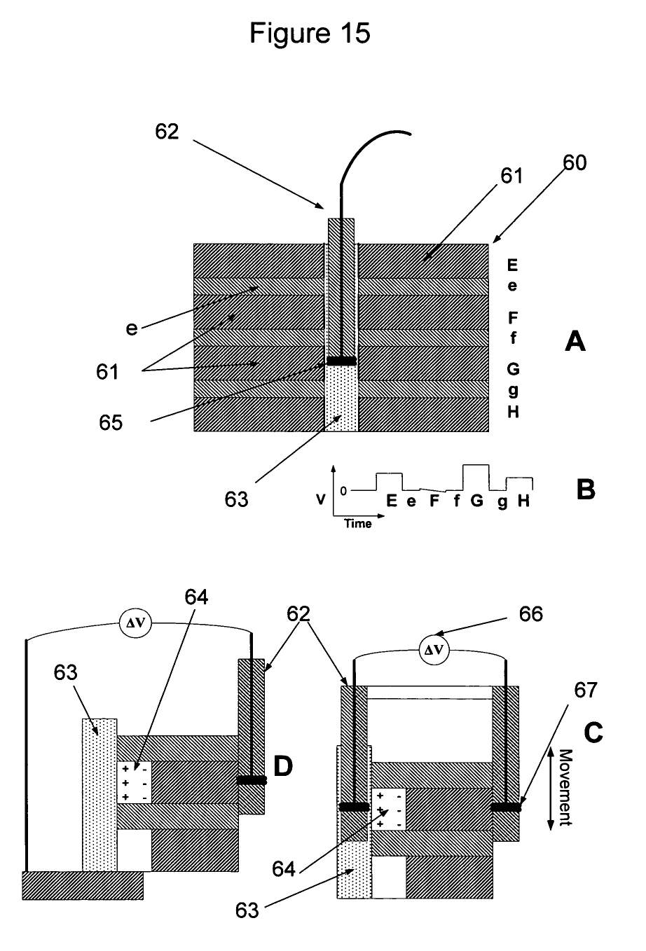

The electric measuring procedure may be envisaged with reference to FIG. 15. FIG. 15A depicts a cross section of a stack (element 60 in FIG. 15A) of PEs (element 61 in FIG. 15A) with the measuring probe (element 62 in FIG. 15A) in the central cavity containing the sample fluid (element 63 in FIG. 15A). The sample fluid is preferably conductive. When a reaction takes place on a perforated element, a potential difference (element 64 in FIGS. 15C and 15D) builds up between different parts of the reacting agent, for example, a potential difference builds up between the part of the reacting agent attached to the electric conducting PE (element 61 in FIG. 15A), and the part facing the central cavity (see FIG. 15C). This potential difference appears between the exposed conducting part of the probe (element 65 in FIG. 15A, i.e., the probe reader) and the corresponding part of the PE that makes contact with the conducting part of the external reader, which moves in correspondence with the internal reader (see FIG. 15C). The potential difference is measured by the potential measuring device (element 66 in FIG. 15C). FIG. 15B depicts an example of the potential difference that may be recorded as the probe is moved in the cavity along the stack of PEs which contains alternate conducting elements (E, F, G, and H in FIG. 15A and insulating elements (e, f, and g in FIG. 15A). When the reactions taking place are fluorescent in nature, and the probe is an optical one, a similar record of light intensity may be observed. FIG. 15D depicts an alternate embodiment of the electric potential reading arrangement. In this embodiment, the potential difference between the external reader (element 67 in FIG. 15C) and a sensor in contact with the fluid filling the central cavity is recorded.

In yet another embodiment of the present invention, reactions that involve changes in the magnetic properties of a material may be studied by replacing the previously described optical or electrical sensors with a magnetic sensor, such as a coil.

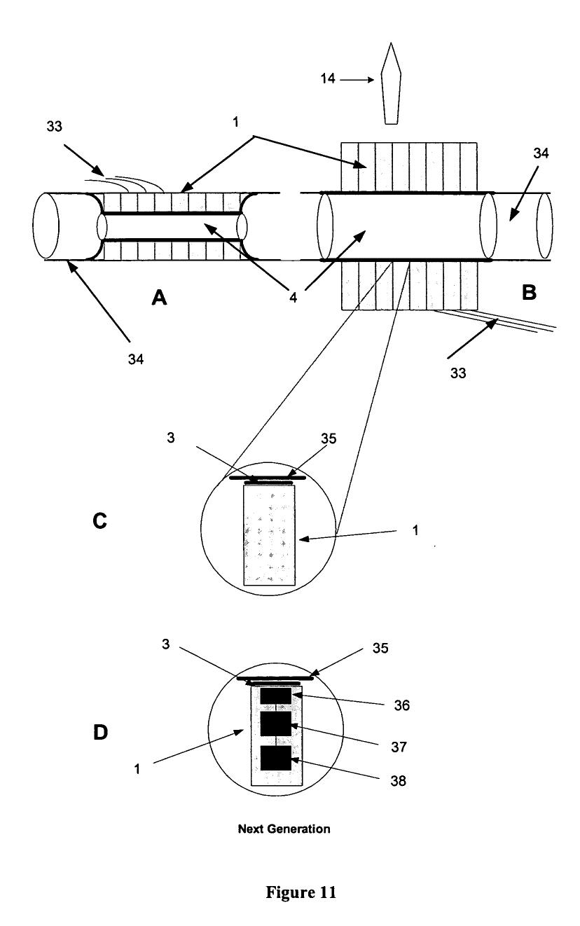

One function that the PE stacks can provide is the ability to monitor fluid composition “in line” in real time (see FIG. 11). The term “in-line” as used herein, defines a system wherein the PE cavity is interposed and continuous with the cavity of a vessel, such as, but not limited to, tubing or a blood vessel, such that the fluid flowing through the vessel continues its flow through the PE cavity that is interposed in the vessel. The fluid, such as, but not limited to, blood, urine, or a fluid associated with an industrial process, may be from a subject, or it may be part of a separate reaction process. The subject is preferably a human, but can be any animal, including a laboratory animal in the context of a clinical trial or screening or activity experiment. Thus, as can be readily appreciated by one of ordinary skill in the art, the present invention is particularly suited to monitoring fluid compositions in any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Since the PE's are assembled as a stack with a central tunnel, continuous uninterrupted flow of a fluid to be monitored through the tunnel may be envisaged. Thus, by using specific active sites along the tunnel, and by applying external reading technology as set forth above, the composition of a fluid flowing through the channel may be monitored. If the reactions occurring at the PE reaction sites are irreversible, the PE stacks of present invention may be used to determine the appearance of a substance, while, if the reactions are reversible, concentration changes of different substances may be continuously monitored.

One embodiment of the present invention, as used for in-line real time monitoring, is described in FIG. 11A. In this embodiment, the PE stack is inserted into any kind of vessel, including, but not limited to an artery, vein, GI tract, pipe, tube, channel, duct, barrel, or other container (element 34 in FIG. 11A). If necessary, a semi-permeable membrane (element 35 in FIG. 11C) may be placed on the active site (element 3 in FIG. 11C) to provide a protective “no clot” or inert surface. The fluid to be monitored flows through the central tunnel (element 4 in FIG. 11A), thus making contact with the different active sites. Activation of the sites occurs when a specific substance reacts with its probe on the active site, thereby creating a signal that can be read, for example, by optical or electrical means. In order to enable external reading, the perforated elements may be transparent (for optical reading) or conductive (for electrical reading). Electrical leads or optical fibers (element 33 in FIG. 11) leading away from the inserted PE stack may be used to guide the triggered signal to sensors and processors, which can be either internal or external. This communication can be aided by conventional wireless transmission means.

In yet another in-line monitoring embodiment (FIG. 11B), the vessel through which the fluid to be monitored flows, is inserted into the central tunnel of the stack (element 4 in FIG. 11B). The PE's thus embrace the monitored tube, while fluid flows through the monitored tube. A semi-permeable membrane (element 35 in FIG. 11C), placed between the monitored vessel and the active sites (element 3 in FIG. 11C), may be used to provide a protective, inert or “no clot” surface, which allows certain substances to reach the active sites (FIG. 11C). The perforated elements may be transparent or conductive in order to enable external reading of the triggered signals from the active sites by optical or electrical means. Again, electrical leads or optical fibers guide the signals from the PE's to sensors and processors, which may be either internal or external.

Another embodiment is illustrated in FIG. 11D. In this embodiment the sensors (element 36 in FIG. 11D), the amplifiers and processors (element 37 in FIG. 11D), and the telemetry transmitters (element 38 in FIG. 11D) are all an integrated part of the Perforated Element.

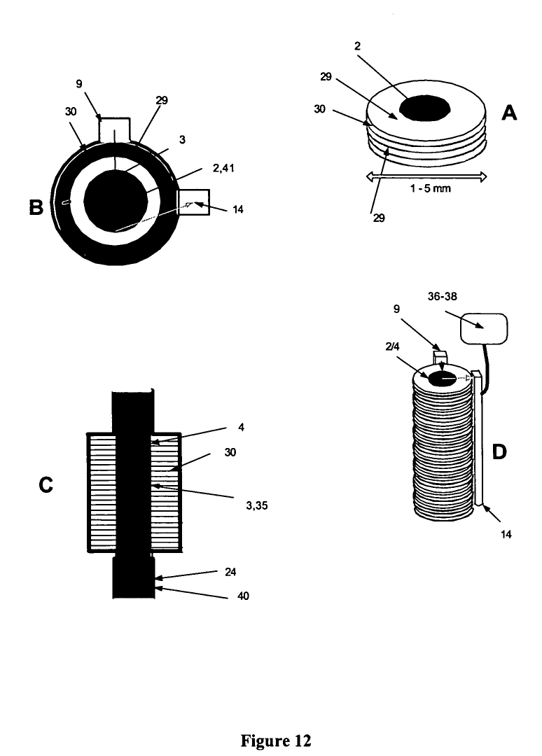

As discussed above, the in line real time system may be used for monitoring fluids inside a subject. In this embodiment, a PE stack is implanted in the subject whose fluids are to be monitored. The subject is typically a human, but is not limited thereto. The stack may be implanted in a vessel such as, but not limited to, an artery, a vein, GI tract, urethra, lymph vessels, and/or thoracic duct (see FIGS. 11 and 12). The PE stack size is designed to fit within the monitored vessel.

In order to enable external reading, the implanted PE's are made either from a transparent substance, for optical sensing, or from a conductive\insulating material, for electrical sensing (see FIG. 12A). In this embodiment, the fluid to be tested, such as blood (element 41 in FIG. 12A), flows through the central tunnel (element 4 in FIG. 12A) of the stack making contact with the different active sites (element 3 in FIG. 12A). In this embodiment, the reading probes must be biocompatible. The active sites are designed to react with specific substances in the blood flow, selected apriori for monitoring. The reaction detection method may also be optical (including fluorescent) or electrical. A semi-permeable membrane (element 35 in FIG. 12C) may be placed between the active sites and the blood flow. Element 40 in FIG. 12C depicts the blood vessel wall. For reading the triggered signals from the active sites, an excitation path and a reading path may be used in conjunction with a stimulator (illuminator/electrical) (element 9 in FIGS. 12B and 12D) and a scanner (element 14 in FIGS. 12B and 12D). Amplification and processing of the signals may be performed at this stage. The detected signals are then transmitted outside the body for further processing and evaluation.

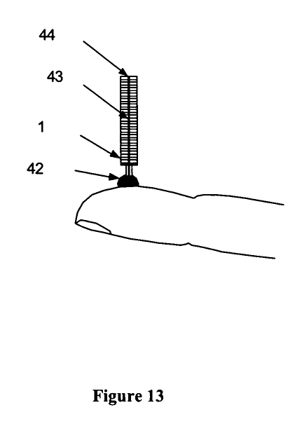

Another embodiment of the present invention is a multi testing diagnostic stack (MTDS, see FIG. 13). In this embodiment, capillary forces are used to suck a drop of blood urine or any other fluid sample (element 42 in FIG. 13) in to the central capillary tunnel (element 43 in FIG. 13) of the multi testing diagnostic stack. The open end of the capillary tunnel is element 44 in FIG. 13. The MTDS is constructed of PE's (element 1 in FIG. 13) with specific active sites, selected according to the diagnostic task or tests to be performed. The tested drop of fluid reacts within the central tunnel of the MTDS. An external sensor (optical, electrical, etc.) may be used to directly read the reactions from the different active sites within the stack. This embodiment provides significant advantages for performing routine blood and urine testing. A very small amount of sample can be used to conduct a large number of tests. Furthermore, a single unit (i.e., a single MTDS) may be used for acquiring the sample as well as performing the variety of required tests. The MTDS thus avoids the cumbersome handling procedures typically associated with standard multi-tests performed today. In addition, the test results may be obtained in most cases almost immediately. The procedure of the present invention also minimizes blood handling, thereby reducing the possibility of blood contamination and transmission of diseases.

As can clearly be seen, the advantages of the present invention are significant. Expensive, mechanically complicated, high resolution, [x,y] scanning systems, used with micro-arrays, may be replaced by simple, inexpensive, reading apparatus. Additionally, the methods of the present invention do not require the ability to approach a specific [x,y] coordinate with high precision either for preparing the site or for reading and analyzing it. The present invention may be implemented in numerous applications where micro arrays are currently used. By using the perforated elements described herein as building blocks for the multi reaction stacks, a tailor-made reaction site structure may easily be prepared, for use in a variety of applications. The present invention thus offers a modular, inexpensive, and efficient system.

The present invention is not to be limited in scope by the specific embodiments described herein. Indeed, various modifications of the invention in addition to those described herein will become apparent to those skilled in the art from the foregoing description and the accompanying Figures. Such modifications are intended to fall within the scope of the appended claims.

Various patents, patent applications, and publications cited herein are incorporated by reference in their entireties for all purposes.

1. A solid support comprising a plurality of individual perforated elements, wherein each perforated element contains one or more perforations; and one or more immobilized molecules attached to the surface of at least one of the perforations within each perforated element;
wherein the plurality of perforated elements are assembled to form a column of perforated elements, and the perforations within each column are aligned so as to form one or more tunnels throughout the column.

2. The solid support of claim 1, wherein the immobilized molecule on each perforated element is different.

3. The solid support of claim 2, wherein the molecule is selected from the group consisting of nucleic acids, carbohydrates, oligonucleotides, peptides, peptideomimetics, pharmacores, biosensors, antibodies, and tumor markers.

4. The solid support of claim 1, wherein the perforated element has a regular geometric shape or an irregular geometric shape with reference to its central axis.

5. The solid support of claim 4, wherein the regular geometric shape is selected from the group consisting of an equilateral triangle, a square, a hexagon, an octagon, and a circle.

6. The solid support of claim 4, wherein the irregular geometric shape is selected from the group consisting of an isosceles triangle, a scalene triangle, a rectangle, a rhombus, a parallelogram, and an irregular trapezoid.

7. The solid support of claim 1, wherein each individual perforated element is constructed of a material selected from the group consisting of organic polymers, glass, metal, a semiconductor, silica, cellulose derivatives, ceramics, and mixtures thereof.

8. The solid support of claim 7, wherein the organic polymer is selected from the group consisting of polystyrene, polycarbonates, polymethylmethacrylate, polyurethanes, polyethylene, polyacrylamides, polypropylene, copolymers of styrene and acrylic acid, copolymers of styrene and methyl methacrylate, and mixtures thereof.

9. The solid support of claim 1, further comprising one or more perforated spacer elements.

10. A solid support library comprising a plurality of solid supports of claim 1.

11. A method for preparing a solid support, comprising the step of
(a) assembling a plurality of perforated elements having different coatings so as to form one or more columns wherein each column comprises a plurality of perforated elements having different coatings.

12. The method of claim 11, wherein the plurality of perforated elements used to build the one or more columns of the solid support are prepared by a process comprising the steps of:
(a) forming a plurality of preparatory stacks, wherein each preparatory stack comprises a plurality of uncoated perforated elements stacked to create a column; and
(b) coating all the perforated elements within a single preparatory stack with the same coating, wherein each individual preparatory stack is coated with a different coating, thereby creating a plurality of preparatory stacks each having a different coating.

13. The method of claim 11, further comprising the step of
assembling one or more spacer perforated elements within one or more of the columns containing different coated perforated elements.

14. A method for identifying a molecule of interest, which method comprises monitoring changes in an immobilized molecule on each perforated element within the solid support of claim 1, which changes result from a chemical, biochemical, or biological process involving the immobilized molecule and the molecule of interest.

15. A method for identifying a molecule of interest, which method comprises monitoring changes in an immobilized molecule on each perforated element within the solid support library of claim 10, which changes result from a chemical, biochemical, or biological process involving the immobilized molecule and the molecule of interest.

16. The method of claim 14, wherein the chemical, biochemical, or biological process is monitored using a probe, which is external or internal to the solid support.

17. The method of claim 16, wherein the probe is an optical, electrical, or magnetic probe moved through the internal cavity created within the solid support.

18. The method of claim 15, wherein the chemical, biochemical, or biological process is monitored using a probe, which is external or internal to the solid support.

19. The method of claim 18, wherein the probe is an optical, electrical, or magnetic probe moved through the internal cavities created by the solid support library.

20. The method of claim 14, wherein the molecule of interest is moved through the cavity formed within the solid support, wherein contact is made between the molecule of interest and the immobilized molecule.

21. The method of claim 15, wherein the molecule of interest is moved through the cavities formed within the solid support library, wherein contact is made between the molecule of interest and the immobilized molecules.
